(19) United States
(12) Patent Application Publication
(10) Pub. No.: US 2003/0105034 A1
(43) Pub. Date: Jun. 5, 2003

(54) Pyridine carboxy derivatives and an aminosugar

(75) Inventor: Morten Sloth Weidner, Virum (DK)

Correspondence Address: BIRCH STEWART KOLASCH & BIRCH, PO BOX 747, FALLS CHURCH, VA, 22040-0747, US

(73) Assignee: Astion Deveopment A/S, Copenhagen (DK)

(21) Appl. No.: 10/251,360

(22) Filed: Sep. 21, 2002

Related U.S. Application Data
(63) Continuation-in-part of application No. 10/187,279, filed on Jun. 28, 2002.
(60) Provisional application No. 60/303,297, filed on Jul. 5, 2001.

(51) Int. Cl. A61K 31/7052; A61K 31/7008; C07H 5/06

(52) U.S. Cl. 514/42; 514/62; 536/18.7

(57) ABSTRACT

The present invention relates to chemical complexes consisting of a pyridine carboxy derivative and an aminosugar as well as pharmaceutical compositions and dietary supplements comprising such complexes. The invention further relates to the use of such compositions or complexes for the preparation of a medicament or a dietary supplement in the suppression of hypersensitivity and inflammatory reactions such as dermatological disorders or to a method of treating such disorders by administering such compositions and complexes to a mammal, such as a human.

### FIELD OF THE INVENTION

The present invention relates to a chemical complex comprising a pyridine carboxy derivative and an aminosugar. The invention further relates to the combined therapeutic activity of a pyridine carboxy derivative and an aminosugar in the suppression of hypersensitivity and inflammatory reactions such as dermatological disorders. The complex of a pyridine carboxy derivative and an aminosugar may also be used as a dietary supplement.

### BACKGROUND OF THE INVENTION

Hypersensitivity is defined as a state of altered reactivity in which the body reacts with an exaggerated immune response to a substance (antigen). Hypersensitivity may be caused by exogenous or endogenous antigens. Hypersensitivity reactions underlie a large number of diseases. Among these, allergic and autoimmune conditions are of great importance. A classification of hypersensitivity diseases is given in the textbook Clinical Medicine (Kumar, P. and Clark, M.: “Clinical Medicine”, 3rd edition, p. 147-150, 1994, Bailliere Tindall, London).

Type I hypersensitivity reactions (IgE mediated allergic reactions) are caused by allergens (specific exogenous antigens), e.g. pollen, house dust, animal dandruff, moulds, etc. Allergic diseases in which type I reactions play a significant role include asthma, eczema (atopic dermatitis), urticaria, allergic rhinitis and anaphylaxis.

Type II hypersensitivity reactions are caused by cell surface or tissue bound antibodies (IgG and IgM) and play a significant role in the pathogenesis of myasthenia gravis, Good-pasture's syndrome and Addisonian pernicious anaemia.

Type III hypersensitivity reactions (immune complex) are caused by autoantigens or exogenous antigens, such as certain bacteria, fungi and parasites. Diseases in which type III hypersensitivity reactions play a significant role include lupus erythematosus, rheumatoid arthritis and glomerulonephritis.

Type IV hypersensitivity reactions (delayed) are caused by cell or tissue bound antigens. This type of hypersensitivity plays a significant role in a number of conditions, e.g. graft-versus-host disease, leprosy, contact dermatitis and reactions due to insect bites.

Type I to type IV hypersensitivity reactions are all classically allergic reactions, which may lead to histamine release. However, hypersensitivity reactions are also those, where histamine release is triggered through the directly action of “triggering substances” with the cellular membrane. Examples of “triggering substances” are, but not limited to, toxins, food constituents and certain drugs.

A number of drug classes are available for the treatment of hypersensitivity reactions. Among these, the corticosteroids are some of the most widely used drugs. Corticosteroids primarily exert their pharmacological action by non-selectively inhibiting the function and proliferation of different classes of immune cells resulting in suppression of hypersensitivity reactions. Unfortunately, the corticosteroids are associated with a number of serious side effects, e.g. immunosuppression, osteoporosis and skin atrophy.

Cancer is caused by an uncontrolled proliferation of cells that express varying degrees of fidelity to their precursors. These cancer cells form a malignant tumour that enlarges and may spread to adjacent tissues or through blood and lymph systems to other parts of the body. There are numerous forms of cancer of varying severity. For most types of cancer there is no effective treatment today.

Aminosugars are the building blocks for the in vivo generation of glycosaminoglycans, formerly known as mucopolysaccharides. Glycosaminoglycans are constituents in various tissues in numerous mammals, both vertebrates and invertebrates and important examples of glycosaminoglycans are the chondroitin sulfates and the keratan sulfates in connective tissue, the dermatan sulfates in skin tissue, and hyaluronic acid in skin tissue and synovial joint fluid.

Administration of aminosugars or glycosaminoglycans in pharmacological doses to individuals suffering from osteoarthritis has resulted in some relief of symptoms and nowadays the use of aminosugars as chondroprotective agents is widely recognised. For example, the oral administration of glucosamine sulfate for alleviating pain and joint mobility is disclosed in the scientific paper of Meletis, C. D, entitled “Natural Medicine approaches for the treatment of degenerative arthritis” (in Alternative and complementary Therapies, Mary Ann Liebert, Larchmont, N.Y., US, vol. 5, no. 3, 1999, pages 136-139). Furthermore, oral administration of glucosamine sulfate for relieving the symptoms of OA is disclosed (see Gaby, A, R: “Natural Treatments for Osteoarthritis”, Alternative medicine review, Thorne Research Inc., Sandpoint, US, vol. 4. no. 5, 199, pages 330-341). Niacinamide, which is also known as nicotinamide, has been found to be a potent inhibitor of poly(ADP-ribose)polymerase. Poly(ADP-ribose)polymerase, also known as poly(ADP-ribose)synthetase or poly(ADP-ribose)transferase is an nuclear enzyme that catalyses the posttranslational modification of nuclear proteins by covalent attachment of ADP-ribosyl moieties derived from NAD⁺ with an accompanying release of nicotinic acid amide. Preferred acceptor proteins are nuclear histones, whose poly-ADP-ribosylation induces local alterations in the architecture of chromatin domains. Inhibitors of poly(ADP-ribose)polymerase have been found to suppress hypersensitivity reactions and inflammation. For example, it is found that niacinamide in a dose of 500 mg six times a day is useful for treating degenerative arthritis (Meletis, C. D, Natural Medicine approaches for the treatment of degenerative arthritis, Alternative and complementary Therapies, Mary Ann Liebert, Larchmont, N.Y., US, vol. 5, no. 3, 1999, pages 136-139). Moreover, niacinamide is effective in increasing joint motility in patients suffering from OA (see Gaby, A, R: “Natural Treatments for Osteoarthritis”, Alternative medicine review, Thorne Research Inc., Sandpoint, US, vol. 4. no. 5, 199, pages 330-341).

Glucosamines, niacin or niacinamide are widely used for various purposes. For example they may form part of orally administered dietary supplements for reducing the pain in joints or muscles. Such compositions comprise a source of phenylalanine (see GB 2 286 528). In addition, glucosamines, niacin or niacinamide is in the form of a comestible together with antioxidants, vegetable extracts, vitamins, amino acids, minerals, herbal extracts, cholinergic complexes, and enzymes. Such a comestible is intended for use in supplementing nutritional deficiencies (See U.S. Pat. No. 5,895,652). Furthermore, an athletic drink comprising niacin in a dose corresponding to the recommended daily requirement and 2000 to 4000 parts of glucosamine together with other sugars and vitamins is disclosed (See EP 0 652 012).

The combination of aminosugars and niacinamide is also included in multi-component compositions for use in treating skin conditions. For example, the U.S. Pat. No. 5,804,594 relates to compositions comprising the essential constituents: a sugar compound that is converted to a glycosaminoglycan in vivo, an antioxidant, at least on amino acid and a transition metal. Such compositions may further comprise a glucosamine, a chondroitin, and vitamin B3 together with a catechin-based preparation, amino acids, a vitamin E source, quercetin dihydrate (a bioflavonoid), pyridopxal 5 phosphate-Co vitamin B6, a methionine source and a vitamin A source. The skin conditions relate to wrinkles, fine lines, thinning, reduced skin elasticity, reduced skin moisture, spider veins, senile purpura, sun damaged skin, aged skin or rough skin.

Moreover, the combination of a pyridine carboxy derivative and aminosugar derivatives of some oligo and polysaccharides for the treatment of hypersensitivity and inflammatory diseases are disclosed in WO 01/74781.

### SUMMARY OF THE INVENTION

The present inventor has found that a combination of niacinamide and an aminosugar has immunomodulating activities and significantly suppresses inflammatory reactions and hypersensitivity in mammals. Such a combination is advantageously provided in the form of a chemical complex consisting of one or more optionally substituted pyridine carboxy derivative(s) or salt(s) thereof and one or more optionally substituted aminosugar(s) or salt(s) thereof. Obviously, the combination may also be provided in the form of a pharmaceutical composition, a dietary supplement or a cosmetic. As was further recognised by the present inventor, the aminosugar according to the present invention may be an aminosugar derivative of a mono-saccharide or an oligosaccaride containing at the most of six saccharide units.

Thus, the present inventor has recognised the therapeutic activity of a combination of one or more optionally substituted pyridine carboxy derivative(s) or salt(s) thereof and one or more optionally substituted aminosugar(s) or salt(s) thereof, for which reason the said combination may be regarded as an active therapeutic agent.

Contrarily to existing therapeutic agents, such as corticosteroids or non-steroidal anti-inflammatory drugs, the chemical complexes and compositions according to the present invention have the advantage of not being likely to be associated with any serious side effects, as all of their components are known to living organisms and acknowledge as non-toxic and well-tolerated by the organism. The present inventor puts forward the hypothesis that the very beneficial therapeutic index exhibited by the complex and compositions comprising said complex according to the invention is superior to the use of the individual constituents of the complex, and this is due to synergistic effects and a lower toxic load.

Accordingly, the present invention provides in a first aspect a chemical complex consisting of:

i) one or more optionally substituted pyridine carboxy derivative(s) or salt(s) thereof according to formula I:

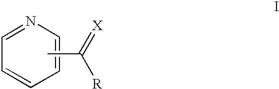

wherein X is selected from O and S; R is selected from OH; OR′; NH₂; NHR′; NR′R″, O⁻Y⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and

ii) one or more optionally substituted aminosugar(s) or salt(s) thereof,

wherein the one or more optionally substituted aminosugar(s) is/are aminosugar derivative(s) of a mono-saccharide or an oligo-saccharide containing of at the most of six saccharide units.

A further aspect of the invention relates to a composition comprising:

i) one or more optionally substituted pyridine carboxy derivative(s) or salt(s) thereof according to formula I;

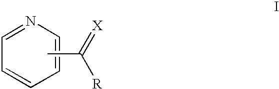

wherein X is selected from O and S; R is selected from OH; OR′; NH₂; NHR′; NR′R″, O⁻Y⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and

ii) one or more optionally substituted aminosugar(s) or salt(s) thereof; and

iii) one or more acceptable excipient(s) or carrier(s),

wherein the one or more optionally substituted aminosugar(s) is/are aminosugar derivative(s) of a mono-saccharide or an oligo-saccharide containing of at the most of six saccharide units.

The chemical complexes and pharmaceutical compositions according to the invention may be employed for various therapeutic applications related to inflammation or hypersensitivity such as treatment of inflammatory skin diseases; treatment of IgE mediated allergic reactions and conditions; treatment of autoimmune disorders; treatment of chronic inflammatory diseases; alleviation of pain; and treatment of cancer.

An important aspect of the invention relates to the use of a combination of one or more optionally substituted pyridine carboxy derivative(s) or salt(s) thereof according to formula I and one or more optionally substituted aminosugar or salt(s) thereof for the preparation of a product for the suppression of hypersensitivity and/or suppression of inflammatory reactions in a mammal, such as a human, as well as to a method for method for suppression of hypersensitivity and suppression of inflammatory reactions in a mammal, comprising the administration to said mammal of an effective amount of a combination of one or more optionally substituted pyridine carboxy derivative(s) or salt(s) thereof and one or more optionally substituted aminosugar(s) or salt(s) thereof, or a chemical complex comprising said combination.

Still further aspects of the invention relate independently to a method for the treatment of hypersensitivity skin disease in a mammal; a method for the treatment of rheumatic conditions; a method for the treatment or prevention of IgE mediated allergic reaction and/or condition in a mammal; a method for the treatment of an autoimmune disease and/or a chronic inflammatory disease in a mammal; a method for the alleviation of pain in a mammal, and a method for the treatment or prevention of cancer in a mammal each method comprising the administration to said mammal of an effective amount of a combination of one or more optionally substituted pyridine carboxy derivative(s) or salt(s) thereof and one or more optionally substituted aminosugar(s) or salt(s) thereof, or a chemical complex comprising said combination.

### DETAILED DESCRIPTION OF THE INVENTION

The present inventor provides data herein indicating that a combination of a glucosamine and niacinamide or a combination of N-acetylglucosamine and niacinamide inhibits inflammation in a dose-dependtly manner. This was tested using the tetradecanoyl phorbol acetate (TPA) induced ear inflammation test model in mice, which is a commonly employed method for screening and evaluation of anti-inflammatory agents. The inhibition observed, was comparable to that of therapeutically relevant doses of (0.1% hydrocortisone 17-butyrate).

Moreover, evidence for a synergistic or additive effect is also provided. The two compounds niacinamide and N-acetylglucosamine, as well as a complex of the two compounds were tested for anti-inflammatory activity in the tetradecanoyl phorbol acetate (TPA) induced ear inflammation test in mice. As can be seen from Example 241, the complex significantly inhibited ear swelling in the same order as the positive control hydrocortisone 17-butyrate, while the individual compounds, niacinamide and N-acetylglucosamine, showed much lesser inhibition of ear swelling.

Surprisingly, the present inventor found that complexes and compositions according to the invention, when applied topically as a cream, was effective in reducing the symptoms of various dermatological disease. As can be seen from example 240, the said complexes and compositions are effective alleviating the symptoms seen in senile pruritus, keloids on the arms and the chest, such as sore and itching, psoriasis and seborrhoeic dermatitis.

Consequently, the combination of one or more optionally substituted pyridine carboxy derivative(s) or salt(s) thereof and one or more optionally substituted aminosugar(s) or salt(s) thereof is effective in suppressing hypersensitivity and inflammatory reactions, in particularly with respect to dermatological diseases.

According to the invention, the combination of one or more optionally substituted pyridine carboxy derivative(s) or salt(s) thereof and one or more optionally substituted aminosugar(s) or salt(s) thereof may be provided in the form of a chemical complex, in the form of a composition comprising said complex and optionally pharmaceutically acceptable excipient(s), or in the form of a pharmaceutical composition comprising the combination of the of one or more optionally substituted pyridine carboxy derivative(s) or salt(s) thereof and one or more optionally substituted aminosugar(s) or salt(s) thereof. Moreover, the one or more optionally substituted pyridine carboxy derivative(s) or salt(s) thereof and the one or more optionally substituted aminosugar(s) or salt(s) thereof may each be provided in separate compositions.

Without being limited to a particular theory, advantageously, said combination is provided in the form of a chemical complex for purposes of achieving a homogeneous mixture of the two agents, which may positively affect the resulting therapeutic effect.

Such chemical complexes are novel and provide a surprisingly effective anti-hypersensitivity and anti-inflammatory effect with a surprisingly good safety profile. Thus the chemical complexes or compositions of the invention are virtually non-toxic and yet very therapeutically effective.

The present inventor proposes the hypothesis that the very advantageous therapeutic index of said combinations of the one or more optionally substituted pyridine carboxy derivative(s) or salt(s) thereof and the one or more optionally substituted aminosugar(s) or salt(s) thereof in comparison to their individual anti-inflammatory effect is due to the synergistic effects between the components of the compositions. Therefore, lower doses may be needed for providing the therapeutic effect, resulting in a lower toxic load on the body in comparison to the individual compound, while still achieving a surprisingly good therapeutic effect.

Accordingly, the present invention provides in a first aspect a chemical complex consisting of:

i) one or more optionally substituted pyridine carboxy derivative(s) or salt(s) thereof according to formula I

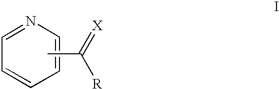

wherein X is selected from O and S; R is selected from OH; OR′; NH₂; NHR′; NR′R″, O⁻Y⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and

ii) one or more optionally substituted aminosugar(s) or salt(s) thereof,

wherein the one or more optionally substituted aminosugar(s) is/are aminosugar derivative(s) of a mono-saccharide or an oligo-saccharide containing of at the most of six saccharide units.

The term “chemical complex” is intended to include the definition defined by IUPAC that read as follows:

“A molecular entity formed by loose association involving two or more component molecular entities (ionic or uncharged), or the corresponding chemical species. The bonding between the components is normally weaker than in a covalent bond.” (IUPAC Compendium of Chemical Terminology 2nd Edition (1997))

Thus, the term “chemical complex” is intended to mean any combination of the component molecules. It is not intended necessarily to implie an ionic or otherwise association between the components. Also as used herein, the chemical complex of the present invention relates to a complex obtainable from the combining of one or more optionally substituted pyridine carboxy derivative(s) or salts thereof and one or more optionally substituted aminosugar or salts thereof.

The complexes of the invention may be prepared according to a number of different methods, which are obvious to a person skilled in the art. The following procedures are non-limiting examples of such methods:

The components of the complex, dosed in appropriate amounts to give the correct molar ratio between the moieties, are dissolved, dispersed, or suspended in an appropriate solvent, for example water, an organic solvent or mixtures thereof. Non-limiting examples of suitable organic solvents are ethanol, methanol, iso-propyl alcohol, acetone, hexane, ethylacetate or mixtures thereof.

The solvent is then removed by a technique suitable for the complex, for example evaporation, in vacou evaporation, spray drying, freeze-drying, fluid bed drying or spin flash drying. Alternatively the complex may be obtained by precipitation and subsequent centrifugation or filtering.

The chemical complexes or compositions of the invention provide pharmacological effects upon administration to the living organism such as immunomodulation, suppression of skin hypersensitivity reactions, suppression of IgE mediated allergic reactions, suppression of autoimmune reactions, reduction of pain, and suppression of cancer;

Accordingly, the present invention relates to a composition comprising:

i) one or more optionally substituted pyridine carboxy derivative(s) or salt(s) thereof according to formula I;

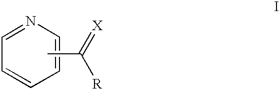

wherein X is selected from O and S; R is selected from OH; OR′; NH₂; NHR′; NR′R″, O⁻Y⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and

ii) one or more optionally substituted aminosugar(s) or salt(s) thereof; and

iii) one or more acceptable excipient(s) or carrier(s),

wherein the one or more optionally substituted aminosugar(s) is/are aminosugar derivative(s) of a mono-saccharide or an oligo-saccharide containing of at the most of six saccharide units.

The term “optionally substituted” is intended to mean the substitution of one or more hydrogen atoms, which is substituted with another atom, chemical group or entity, termed substituents. Illustrative examples of substituents include carboxyl, formyl, amino, hydroxyl, halogen, nitro, sulphono, sulphanyl, C₁₋₆-alkyl, aryl, aryloxy, aryloxycarbonyl, arylcarbonyl, heteroaryl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)-amino-C₁₋₆-alkyl-aminocarbonyl, C₁₋₆-alkylcarbonylamino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, C₁₋₆-alkoxyl, oxo, C₁₋₆-carboxyl, C₁₋₆-alkoxycarbonyl, C₁₋₆-alkylcarbonyl, where aryl and heteroaryl representing substituents may be substituted 1-5 times with C₁₋₆-alkyl, C₁₋₆-alkoxy, nitro, cyano, hydroxy, amino or halogen. In general, the above substituents may be susceptible to further optional substitution.

The term “halogen” includes fluorine, chlorine, bromine and iodine.

The term “C₁-C₂₀ alkyl” is intended to mean a linear or branched saturated hydrocarbon chain wherein the longest chains has from one to twenty carbon atoms, such as methyl, ethyl, n-propyl, isopropyl, n-butyl, isobutyl, sec-butyl, tert-butyl, pentyl, isopentyl, neopentyl, hexyl, heptyl, octyl, undecacyl, dodecyl, etc. A branched hydrocarbon chain is intended to mean a C₁-C₂₀ alkyl substituted at any carbon with a hydrocarbon chain. The C₁-C₂₀ alkyl chain of the present invention may be optionally substituted.

The term “C₂-C₂₀ alkenyl” is intended to mean a linear or branched unsaturated hydrocarbon chain with one or more double bindings and wherein the longest chains has from one to twenty carbon atoms. A branched hydrocarbon chain is intended to mean a C₁-C₂₀ alkyl substituted at any carbon with a hydrocarbon chain. The C₂-C₂₀ alkenyl chain of the present invention may be optionally substituted.

The term “C₁-C₂₀ alkoxyl” is intended to mean a linear or branched hydrocarbon chain wherein the longest chains has from one to twenty carbon atoms, such as methoxy, ethoxyl, n-propoxyl, isopropoxyl, n-butoxyl, isobutoxyl, isopentoxyl, hexoxyl, heptoxyl, octoxyl, etc. A branched hydrocarbon chain is intended to mean a C₁-C₂₀ alkyl substituted at any carbon with a hydrocarbon chain. The C₁-C₂₀ alkyl chain of the present invention may be optionally substituted.

In the present invention, the term “aminosugar” is intended to mean one or more amino derivatives of a monosaccharide (aldoses and ketoses) and its corresponding sugar alcohols (alditols) such as trioses, tetroses, pentoses, hexoses, heptoses and octoses. The aldose, ketose, or alditol has one or more hydroxy groups replaced by any amino group at any position, including the anomeric position. An aminosugar is thus a deoxyamino derivative of an aldose, ketose, or alditol. The term is also intended to mean polyamino sugars, wherein more than one hydroxy group has been replaced by an amino group (e.g. dideoxydiamino-, trideoxytriamino-derivatives).

The term “aminosugar” is also intended to mean amino derivatives of di and oligo-saccharides comprising at least one of said monosaccharides and at most six of said mono-saccharides. Consequently, in the case of such di and oligo-saccharides, the amino group may be position of glycosidation. Suitably, the amino group may not be the position of glycosidation.

An amino group of an aminosugar may be alkylated, arylated or acylated or, alternatively, present as its free amine form (NH₂). Similarly, the hydroxyl groups may be optionally protected or derivatised such as alkylated, arylated or acylated or, alternatively, present in its free hydroxyl form.

The amine of the amino sugar may exist as ammonium salt, such as its quaternary ammonium salt, using organic or mineral acids, as is known to the person skilled in the art. Furthermore, other functional groups on the aminosugar may be in the form of a salt. Similarly, prodrug derivatives of the aminosugar are anticipated by the present inventor. The prodrug form may be the result of the derivatisation of the amino group or another functional group present on the aminosugar, as is known to the person skilled in the art.

Furthermore, an aminosugar may have one or more hydroxy groups replaced by any amino group at any position and a further one or more hydroxy groups replaced by a hydrogen (a deoxy sugar), a thiol (a thiosugar), a halogen (a deoxyhalo sugar), an anhydrosugar (a sugar preparable via an intramolecular displacement with a hydroxyl to form an oxirane or oxetane), a carbonyl group.

In a particularly suitable embodiment of the invention, the aminosugar is sulphated or phosphorylated at the anomeric, 2-, 3-, 4-, or 6- position, typically at the 2-, 3-, or 4-position. In another suitable embodiment of the invention the aminosugar is N-acetylated.

Furthermore, a combination of suitable embodiments include the aminosugar sulphated or phosphorylated as well as in its salt form having Na⁺; K⁺; Mg⁺⁺; Ca⁺⁺; or NH₄⁺ as counter ions.

Particularly suitable aminosugars according to the invention are glucosamine, galactosamine or mannosamine, their derivatives and salts thereof, typically glucosamine sulfate, glucosamine hydrochloride, N-acetylglucosamine, galactosamine sulfate, galactosamine hydrochloride, N-acetylgalactosamine, mannosamine sulfate, mannosamine hydrochloride or N-acetylmannosamine. Also other aminosugars known to the person skilled in the art are suitable for use.

As stated the complexes contains one or more optionally substituted pyridine carboxy derivatives of Formula I or salt(s) thereof. It should also be understood that salts of compounds of formula I are anticipated, including, for instance hydrates and solvent addition forms. The term “base addition salts” include alkali metals, such as sodium and potassium, alkali earth metals, such as calcium and magnesium, and organic addition salts such as quaternary ammonium cations.

The chemical complex of the present invention relates to a complex obtainable from the combining of a pyridine carboxy derivative of Formula I and an optionally substituted aminosugar.

As stated, the complex comprises, in part, the optionally substituted pyridine carboxy derivative according to Formula I wherein R may be selected from OH; OR′; NH₂; NHR′; NR′R″, O⁻Y⁺, and halogen. R′ and R″ may independently be selected from optionally substituted C₁-C₂₀ alkyl.

As used herein, the pyridine carboxy derivative includes salts of compounds of formula I. The salts may be any pharmaceutically acceptable salt including hydrates, solvent addition forms, acid addition salts. In different embodiments of the invention, the salt is a hydroiodide, hydrochloride or a hydrobromide, e.g. nicotinamide hydroiodide.

The term “base addition salts” include alkali metals, such as sodium and potassium, alkali earth metals, such as calcium and magnesium, and organic addition salts such as quaternary ammonium cations.

As stated, the complex comprises, in part, the optionally substituted pyridine carboxy derivative according to Formula I wherein R may be selected from OH; OR″; NH₂; NHR″; NR″R′″, O−Y+, and halogen. R″ and R′″ may independently be selected from optionally substituted C₁-C₂₀ alkyl, optionally substituted C₁-C₂₀ alkoxyl and optionally substituted C₂-C₂₀ alkenyl. In suitable embodiments, the carbon chain length of R′ and R″ are shorter than twenty carbon atoms, e.g. from C₁-C₁₀, C₁-C₈, C₁-C₆, C₁-C₄ or C₁-C₃. With respect to the optionally substituted alkenyls, the carbon chain length is at least two carbon. Thus, the optionally substituted alkenyls can have any length, e.g. from C₂-C₁₂ C₂-C₁₀, C₂-C₈, C₂-C₆, C₂-C₄ or C₂-C₃.

The optionally substituted pyridine carboxy derivative, for illustrative purposes, may be selected from the group consisting of optionally substituted nicotinic acid, its corresponding acyl halide, ester, acid salt, or amide, nicotinamide; optionally substituted isonicotinic acid, its corresponding acyl halide, ester, acid salt, or amide, isonicotinamide; and optionally substituted picolinic acid, its corresponding acyl halide, ester, acid salt, or amide, picolinamide.

In the embodiment where the optionally substituted pyridine carboxy derivative is an amide, the amide may be its free primary amide (NH₂), its secondary amide (NHR′) or its tertiary amide (NR′R″).

As stated, the pyridine carboxy derivative may be optionally substituted. In one suitable embodiment, the pyridine carboxy is further substituted with a carboxy group such as a carboxylic acid, acyl halide, carboxylic ester, or acetamide. The pyridine carboxy may be substituted 0 to 4 times, such as 0, 1, 2, 3, or 4 times, preferably 0 to 1 time, most preferably 0 times.

In a preferred embodiment of the invention the pyridine carboxy derivative is selected from the group consisting of niacinamide, nicotinic acid, methyl nicotinate, ethyl nicotinate, N2-methylniacinamide and N2-ethylniacinamide.

In very interesting embodiments of the invention, the pyridine carboxy derivative is pyridine-3-carboxy derivative. Hence, in different embodiments of the invention, the pyridine carboxy derivative is niacinamide, thioniacinamide, 6-aminoniacinamide, N2-methyl-niacinamide, N2-ethyl-niacinamide, nicotinic acid or inosital hexaniacinate or derivatives thereof. As stated above, these pyridine carboxy derivatives may optionally be further substituted or they may be provided as salts. In some embodiments, the pyridine ring may be substituted with an amino group or alkoxy group.

Niacinamide is a derivative of niacin. In a suitable embodiment of the invention, the pyridine carboxy derivative is niacinamide. Niacinamide may be obtained from natural sources or synthetically. However, niacinamide may also be obtained from precursors, that upon chemicall or enzymatic reactions, that either may take in vivo after administering niacinamide or outside the body, releases niacinamide. The pyridine carboxy derivative may be such a precursor, which, upon acetylation by bacteria in the gut lumen or by suitable enzymes in vivo, is converted into niacinamide. The acetylation may also take place in a pharmaceutical formulation containing acetylating bacteria, such as E. Coli bacteria or lactic bacteria. A further precursor of niacinamide may be inositol hexaniacinate, which upon hydrolyses and subsequent acetylation may result in the formation of niacinamide.

As stated the combination of the two kinds of compounds provides a surprisingly effective therapeutic agent for suppression of hypersensitivity and inflammatory reactions. The proper therapeutic efficacy may, in part, be adjusted by providing the two agents in suitable molar ratios or mass ratios.

Hence, the combination of the one or more optionally substituted pyridine carboxy derivative(s) or salt(s) thereof and the one or more optionally substituted aminosugar(s) or salt(s) thereof in a chemical complex or in a compositions according to the invention are present in a molar ratio of between about 1:10000 to 10000:1. Preferably, the molar ratio is of between about 1:1000 to 1000:1 1:100 to 100:1, 1:50 to 50:1, or about 1:40 to 40:1, preferably of about 1:30 to 30:1, such as about 1:25 to 25:1, about 1:20 to 20:1, about 1:18 to 18:1, about 1:16 to 16:1, about 1:14 to 14:1, or about 1:12 to 1:12, more preferably of about 1:10 to 10:1, such as about 1:9 to 9:1, about 1:8 to 8:1, about 1:7 to 7:1, about 1:6 to 6:1, such as from 1:5 to 5:1, such as from 1:4 to 4:1, from 1:3 to 3:1, such as from 1:2 to 2:1, such as 1:1.

Alternatively defined, the ratio between one or more optionally substituted pyridine carboxy derivative(s) or salt(s) thereof and the one or more optionally substituted aminosugar(s) or salt(s) thereof may be expressed as a mass ratio. The mass ratio is of between about 1:10000 to 10000:1. Preferably, the molar ratio is of between about 1:1000 to 1000:1, 1:100 to 100:1, 1:50 to 50:1, or about 1:40 to 40:1, preferably of about 1:30 to 30:1, such as about 1:25 to 25:1, about 1:20 to 20:1, about 1:18 to 18:1, about 1:16 to 16:1, about 1:14 to 14:1, or about 1:12 to 1:12, more preferably of about 1:10 to 10:1, such as about 1:9 to 9:1, about 1:8 to 8:1, about 1:7 to 7:1, about 1:6 to 6:1, such as from 1:5 to 5:1, such as from 1:4 to 4:1, from 1:3 to 3:1, such as from 1:2 to 2:1, such as 1:1.

For the administration to a mammal, such as a human, the chemical complex may be administered directly, eventually provided in a capsule or the like. More convenient, the complex may be formulated into a composition comprising the chemical complex and optionally, one or more acceptable excipients. Alternatively, the combination of the two agents may also be formulated into a composition without being provided as a chemical complex. Thus, in some embodiments of the invention, the chemical complexes or compositions further comprise one of more excipent(s) or carrier(s), preferably pharmaceutically acceptable excipent(s) or carrier(s).

The term “composition” is intended to mean cosmetic compositions, pharmaceutical compositions, nutritional compositions such as food supplements as well as compositions in the field of cosmeceuticals and neutraceuticals.

As stated supra, the combination of the one or more optionally substituted pyridine carboxy derivative of Formula I or salt(s) thereof and the one or more optionally substituted aminosugar(s) or salt(s) thereof possesses significant anti-hypersensitivity and anti-inflammatory activity. Accordingly, said combination is the active agent in compositions for use in the treatment of diseases or disorders associated with inflammation and/or hypersensitivity. For that reason, the compositions of the present invention does not necessarily comprise other compounds than those excipients needed for the formulation of a pharmaceutical or dietary supplement. That is to say that a number of compounds are not considered to add potential benefits to the composition of the invention or to the use according to the present invention of said compositions for the suppression of hypersensitivity and inflammation.

Hence in one embodiment of the invention, the composition consists of one or more optionally substituted pyridine carboxy derivative of Formula I or salt(s) thereof and one or more optionally substituted aminosugar(s) or salt(s) together with one or more acceptable excipient(s) or carrier(s).

Moreover, according to the invention the compositions may be essentially free of dietary constituents that forms part of the daily food intake, e.g. various vitamins, antioxidants, transition metals, minerals and the essential amino acids. Accordingly, in one embodiment the compositions of the invention are essentially free of phenylalanine, such as less than 0.5% w/w, less than 0.3, 0.2 or 0.1% w/w, and if possible they does not contain phenylalanine at all. The presence of phenylalanine may be avoided because of the risk of phenylalanine intolerance. Moreover, the presence of vitamins in the compositions may not add any further suitable therapeutic relevant effect. Thus, in a further embodiment, the compositions of the invention are essentially free of or do not contain ascorbic acid, Vitamin E, Vitamin D, or Vitamin A.

Thus, in suitable embodiments according to the invention, the composition of the invention does not further comprise a source of phenylalanine. In interesting embodiments therof, the compositions do not further comprise ascorbic acid, Vitamin E, Vitamin D, or Vitamin A.

Also importantly, the compositions according to the invention do not comprise aminosugars consisting of more than 6 saccharides units. Therefore, the compositions according to the invention do not comprise both a glucosamine or a chondroitin.

Hence in one embodiment of the invention, the composition consists of one or more optionally substituted pyridine carboxy derivative(s) or salt(s) thereof and one or more optionally substituted aminosugar(s) or salt(s) together with one or more acceptable excipient(s) or carrier(s).

The chemical complexes or compositions of the present invention may be combined with any other therapeutically active agent in order to strengthen, improve, potentiate, or prolong the therapeutic actions of said complexes and said compositions. Thus according to the invention, the composition may further comprise one or more suitable therapeutically active agent, e.g. an agent for treating cancer, an anti-inflamatory agent, an antihistamine or an agent for the relief of pain.

The compositions according to the present invention may be formulated for oral, topical, transdermal, or parenteral administration, preferably oral or topical administration. The compositions according to the present invention may be formulated as a pharmaceutical composition for oral, topical, transdermal, or parenteral administration, preferably oral or topical administration.

In a suitable embodiment of the invention, the compositions are used for oral administration. However, in a most preferred embodiment of the invention the compositions or complexes are used for topical administration.

The optionally substituted pyridine carboxy derivative and the optionally substituted aminosugar may together be comprised in a single formulation or may each individually be comprised in separate formulations. The separate formulations may be administered in a simultaneous or non-simultaneous manner. As stated, the optionally substituted pyridine carboxy derivative and the optionally substituted aminosugar are together comprised in a single formulation.

The active ingredients of the chemical complex or pharmaceutical composition of the present invention need not be administered as one pharmaceutical entity, but may of course be administered as individual compounds or pharmaceutical compositions. In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions for oral, topical, transdermal, or parenteral administration may be in form of, e.g., solid, semi-solid or fluid compositions and formulated according to conventional pharmaceutical practice, see, e.g., “Remington: The science and practice of pharmacy” 20^{th} ed. Mack Publishing, Easton Pa., 2000 ISBN 0-912734-04-3 and “Encyclopedia of Pharmaceutical Technology”, edited by Swarbrick, J. & J. C. Boylan, Marcel Dekker, Inc., New York, 1988 ISBN 0-8247-2800-9.

The choice of pharmaceutically acceptable excipients in a composition for use according to the invention and the optimum concentration thereof is determined on the basis of the selection of pyridine carboxy derivative, selection of the aminosugar, the kind of dosage form chosen and the mode of administration. However, a person skilled in the art of pharmaceutical formulation may find guidance in e.g., “Remington: The science and practice of pharmacy” 20^{th} ed. Mack Publishing, Easton Pa., 2000 ISBN 0-912734-04-3. A pharmaceutically acceptable excipient is a substance, which is substantially harmless to the individual to which the composition will be administered. Such an excipient suitably fulfils the requirements given by the national drug agencies. Official pharmacopeias such as the British Pharmacopeia, the United States, of America Pharmacopeia and the European Pharmacopeia set standards for well-known pharmaceutically acceptable excipients.

For topical, trans-mucosal and trans-dermal compositions, such as administration to the mucosa or the skin, the compositions for use according to the invention may contain conventional non-toxic pharmaceutically acceptable carriers and excipients including microspheres and liposomes.

The topical, trans-mucosal and trans-dermal compositions for use according to the invention include an array of solid, semi-solid and fluid compositions. Compositions of particular relevance are e.g. pastes, ointments, hydrophilic ointments, creams, gels, hydrogels, solutions, emulsions, suspensions, lotions, liniments, resoriblets, suppositories, enema, pessaries, moulded pessaries, vaginal capsules, vaginal tablets, shampoos, jellies, soaps, sticks, sprays, powders, films, foams, pads, sponges (e.g. collagen sponges), pads, dressings (such as, e.g., absorbent wound dressings), drenches, bandages, plasters and transdermal delivery systems.

The pharmaceutically acceptable excipients for topical, trans-mucosal and trans-dermal compositions may include solvents, buffering agents, preservatives, humectants, chelating agents, antioxidants, stabilizers, emulsifying agents, suspending agents, gel-forming agents, ointment bases, suppository bases, penetration enhancers, perfumes, skin protective agents, diluents, disintegrating agents, binding agents, lubricants and wetting agents.

The oral compositions for use according to the invention include an array of solid, semi-solid and fluid compositions. Compositions of particular relevance are e.g. solutions, suspensions, emulsions, uncoated tablets, immediate-release tablets, modified-release tablets, gastro-resistant tablets, orodispersible tablets, efferverscent tablets, chewable tablets, soft capsules, hard capsules, modified-release capsules, gastro-resistant capsules, uncoated granules, effervescent granules, granules for the preparation of liquids for oral use, coated granules, gastro-resistant granules, modified-release granules, powders for oral administration and powders for the preparation of liquids for oral use.

The pharmaceutically acceptable excipients may include solvents, buffering agents, preservatives, humectants, chelating agents, antioxidants, stabilizers, emulsifying agents, suspending agents, gel-forming agents, diluents, disintegrating agents, binding agents, lubricants, coating agents and wetting agents.

Typical solvents may be selected from the group comprising water, alcohols, vegetable or marine oils (e.g. edible oils like almond oil, castor oil, cacao butter, coconut oil, corn oil, cottonseed oil, linseed oil, olive oil, palm oil, peanut oil, poppyseed oil, rapeseed oil, sesame oil, soybean oil, sunflower oil, and teaseed oil), mineral oils, fatty oils, liquid paraffin, polyethylene glycols, propylene glycols, glycerol, liquid polyalkylsiloxanes, and mixtures thereof.

Typical buffering agents may be selected from the group comprising of citric acid, acetic acid, tartaric acid, lactic acid, hydrogenphosphoric acid, diethylamine etc.

Typical preservatives may be selected from the group comprising parabens, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Typical humectants may be selected from the group comprising glycerin, propylene glycol, sorbitol, lactic acid, urea, and mixtures thereof. Typical chelating agents are but not limited to sodium EDTA and citric acid. Typical antioxidants may be selected from the group comprising butylated hydroxy anisole (BHA), ascorbic acid and derivatives thereof, tocopherol and derivatives thereof, cysteine, and mixtures thereof. Suitable emulsifying agents may be selected from the group comprising naturally occurring gums, e.g. gum acacia or gum tragacanth; naturally occurring phosphatides, e.g. soybean lecithin; sorbitan monooleate derivatives; wool fats; wool alcohols; sorbitan esters; monoglycerides; fatty alcohols, fatty acid esters (e.g. triglycerides of fatty acids); and mixtures thereof.

Suitable suspending agents may be selected from the group comprising celluloses and cellulose derivatives such as, e.g., carboxymethyl cellulose, hydroxyethylcellulose, hydroxypropylcellu lose, hydroxypropylmethylcellulose, carrageenan, acacia gum, arabic gum, tragacanth, and mixtures thereof.

Suitable gel bases and viscosity-increasing components may be selected from the group comprising liquid paraffin, polyethylene, fatty oils, colloidal silica or aluminium, zinc soaps, glycerol, propylene glycol, tragacanth, carboxyvinyl polymers, magnesium-aluminium silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Typical ointment bases may be selected from the group comprising beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Typical hydrophobic ointment bases may be selected from the group comprising paraffins, vegetable oils, animal fats, synthetic glycerides, waxes, lanolin, and liquid polyalkylsiloxanes. Typical hydrophilic ointment bases are, but not limited to, solid macrogols (polyethylene glycols).

Suitable powder components may be selected from the group comprising alginate, collagen, lactose, powder, which is able to form a gel when applied to a wound (absorbs liquid/wound exudate).

Suitable diluents and disintegrating agents may be selected from the group comprising lactose, saccharose, emdex, calcium phosphates, calcium carbonate, calcium sulphate, mannitol, starches and microcrystaline cellulose.

Suitable binding agents may be selected from the group comprising saccharose, sorbitol, gum acacia, sodium alginate, gelatine, starches, cellulose, sodium carboxymethylcellulose, methylcellulose, hydroxypropylcellulose, polyvinylpyrrolidone and polyethyleneglycol.

Typical wetting agents may be selected from the group comprising sodium laurylsulphate and polysorbate 80.

Suitable lubricants may be selected from the group comprising talc, magnesium stearate, calcium stearate, silicium oxide, precirol and polyethylenglycol.

Suitable coating agents may be selected from the group comprising hydroxypropylcellulose, hydroxypropylmethylcellulose, polyvinylpropylidone, ethylcellulose and polymethylacrylates.

Typical suppository bases may be selected from the group comprising oleum cacao, adeps solidus and polyethylenglycols.

A dietary supplement is defined according to the U.S. Food and Drug Administration in the Dietary Supplement Health and Education Act of 1994 (DSHEA). The DSHEA gives defines a dietary supplement as “ . . . a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these things” . . . and “is intended for ingestion in pill, capsule, tablet, or liquid form”. Similar definitions exist in other parts of the world, e.g. in Europe. In the present context, the definition is as defined above. Different denominations concerning “dietary supplements” are used around the world, such as “food supplements”, “neutraceuticals”, “functional foods” or simply “foods”. In the present context the term “dietary supplement” covers any such denomination or definition.

The composition comprises an optionally substituted pyridine carboxy derivative according to formula I and an optionally substituted aminosugar as defined for the chemical complexes. Correspondingly, the composition of the present invention may comprise the complex as defined supra. Thus the aminosugar may be selected from the group consisting of glucosamine, galactosamine, derivatives and salts thereof, e.g. wherein the aminosugar is N-acetylglucosamine or N-acetylgalactosamine. A preferred composition comprises N-acetylglucosamine.

Another aspect of the invention relates to the pharmacological effects observed for the chemical complexes and the compositions disclosed by the present invention. It has surprisingly been found that the chemical complex or composition of the invention exhibits an anti-inflammatory effect in the same order as seen for the steroidal anti-inflammatory drug, hydrocortisone 17-butyrate. Moreover, it was demonstrated that the anti-inflammatory effect of the chemical complex or composition of the invention was dose-dependent, thus indicating that the chemical complex or composition has a direct effect on inflammation.

The anti-inflammatory activity was demonstrated in the TPA induced ear inflammation test in mice, which is a commonly employed method for screening and evaluation of antiinflammatory drugs (see Examples).

Thus, in a broad sense the chemical complexes or compositions of the invention provide an anti-hypersensitivity and anti-inflammatory. The present inventor has recognised that a number of diseases or conditions relate to the inflammation provoked in the TPA induced mouse ear oedema test. Such diseases or conditions may be treated by the present complexes and compositions of the invention. In a more specific sense, the chemical complexes or compositions of the invention provides suppression of hypersensitivity reactions, suppression of inflammatory reactions, suppression of cartilage degeneration, suppression of IgE mediated allergic reactions, suppression of autoimmune reactions, reduction of pain, and suppression of cancer.

Given the pharmacological actions of a chemical complex consisting of one or more optionally substituted pyridine carboxy derivative(s) or salt(s) thereof and one or more optionally substituted aminosugar(s) or salt(s) thereof, the use of a combination of one or more optionally substituted pyridine carboxy derivative(s) or salt(s) thereof and one or more optionally substituted aminosugar(s) or salt(s) thereof, of a complex consisting of said combination or a composition comprising said combination for the preparation of a product for the suppression of hypersensitivity and/or suppression of inflammatory reactions in a mammal is a further aspect of the invention.

A further aspect of the invention relates to the use of a complex of the invention for the treatment of autoimmune disorders and IgE mediated allergic conditions. Correspondingly, the invention further relates to method for the treatment or prevention of autoimmune disorders comprising the administration of the chemical complexes or compositions of the invention to a mammal, preferentially a human.

Thus, a further aspect of the invention relates to the treatment of autoimmune disorders such as For illustrative purposes, the treatment of autoimmune disorders relates to the treatment of Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjögren's Disease, Lupus Erythematosus, Primary vasculitis, Rheumatoid Arthritis, Juvenile Arthritis, Mixed Connective Tissue Disease, Psoriasis, Pemfigus, Pemfigoid, and Dermatitis Herpetiformis.

Moreover, a still further aspect relates to a method for suppression of hypersensitivity and suppression of inflammatory reactions in a mammal, comprising the administration to said mammal of an effective amount of a combination of one or more optionally substituted pyridine carboxy derivative(s) or salt(s) thereof and one or more optionally substituted aminosugar(s) or salt(s) thereof, or a chemical complex comprising said combination.

As defined herein, the term “mammal” is intended to include all mammals including a human.

As used herein, the term “effective amount” relates to the effective dose to be determined by a qualified practitioner, who may titrate dosages to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration, or other factors known to a medical practitioner.

As used herein, the “term treatment” relates to treatment of symptoms or prevention the relapse of symptoms in a person diagnosed with a disease related to inflammation, hypersensitivity, infection, cancer and/or pain.

As stated, the chemical complexes or compositions of the invention may provide suppression of hypersensitivity reactions, suppression of inflammatory reactions, suppression of IgE mediated allergic reactions, suppression of autoimmune reactions, reduction of pain, and suppression of cancer.

In one embodiment, the suppression of inflammatory reactions is in the managing dermatological disorder or disease, e.g treatment of atopic dermatitis, contact dermatitis, seborrhoeic dermatitis, pruritus, nodular prurigo (prurigo nodularis hyde), urticaria, acne, rosacea, alopecia, vitiligo and psoriasis.

Thus, in one embodiment the treatment of hypersensitivity, inflammation or cartilage degeneration relates to the treatment of rheumatic disorders, e.g. rheumatoid arthritis, osteoarthritis, ankylosing spondylitis, Reiter's syndrome, psoriastic arthritis, juvenile chronic arthritis, enteropathic synovitis, infective arthritis, soft tissue rheumatism and fibromyalgia. In another embodiment, the hypersensitivity and inflammation relates to the treatment of gout. In an interesting embodiment thereof, the compositions and complexes is for the treatment of muscle pain, e.g. muscle pains in relation to arthritis.

In another embodiment, the suppression of hypersensitivity and/or suppression of inflammatory reactions is/are for the treatment of IgE mediated allergic reactions, such as asthma, eczema (e.g. atopic dermatitis), urticaria, allergic rhinitis and/or anaphylaxis.

As stated, the complexes and compositions according to the invention are of use in the treatment of autoimmune diseases. For illustrative purposes, the treatment of autoimmune disorders relates to the treatment of Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjögren's Disease, Lupus Erythematosus, Primary vasculitis, Rheumatoid Arthritis, Juvenile Arthritis, Mixed Connective Tissue Disease, Psoriasis, Pemfigus, Pemfigoid, and Dermatitis Herpetiformis.

Thus, in one embodiment the treatment of hypersensitivity, inflammation or cartilage degeneration relates to the treatment of rheumatic disorders, e.g. rheumatoid arthritis, osteoarthritis, ankylosing spondylitis, Reiter's syndrome, psoriastic arthritis, juvenile chronic arthritis, enteropathic synovitis, infective arthritis, soft tissue rheumatism and fibromyalgia. In another embodiment, the hypersensitivity and inflammation relates to the treatment of gout. In an interesting embodiment thereof, the compositions and complexes is for the treatment of muscle pain, e.g. muscle pains in relation to arthritis.

The therapeutic action of the complexes and compositions of the invention may be relevant to diseases associated with hypersensitivity reactions or inflammation in general. Accordingly, the chemical complexes or compositions of the invention are suitable for the treatment or prevention of diseases caused by inflammation of various tissues, e.g. inflammation of the prostate, in particular prostatitis. Particularly, the treatment of hypersensitivity relates to the treatment of contact dermatitis, insect bites, allergic vasculitis, post-operative reactions, transplantation rejection (graft-versus-host disease), and so forth.

Furthermore, the complexes and the compositions of the invention may be used for the treatment of cancer. The present inventor puts forward the hypothesis that the anticancer effect is due to a combination of immunomodulating and tumour-suppressing effects of the complexes and compositions of the invention.

The use of a product combining the optionally substituted pyridine carboxy derivative and the optionally substituted aminosugar may be done in an array of manners of administration. The optionally substituted pyridine carboxy derivative and the optionally substituted aminosugar may together be comprised in a single formulation or are each individually comprised in separate formulations.

Furthermore, the manner of administration may be such that the combination is administered in a simultaneous or non-simultaneous manner. Thus, a formulation containing an optionally substituted pyridine carboxy derivative may be administered first and another separate formulation containing an optionally substituted aminosugar may be administered simultaneously or subsequently, or in an opposite order of administration.

However, in a preferred embodiment, the optionally substituted pyridine carboxy derivative and the optionally substituted aminosugar are together comprised in a single formulation.

In a further preferred embodiment, the combination of an optionally substituted pyridine carboxy derivative and an optionally substituted aminosugar is a chemical complex as defined supra.

According to the use of a product combining an optionally substituted pyridine carboxy derivative and an optionally substituted aminosugar, the product may further comprise one or more therapeutically active agents.

Moreover, the product of the invention may be administered by means of oral, topical, transdermal, or parenteral administration, or combinations thereof. However, preferable manners of administration are oral and/or topical administration.

### EXAMPLES

The following examples describe the preparation of chemical complexes of the present invention.

### General Method Examples 1-226

The pyridine carboxy derivative and the aminosugar derivative are dissolved in as little water as possible and the solvent is removed by spray drying or freeze-drying. After the solvent is removed the product is a white to yellowish powder.

The powder is suitable for any type of product e.g. pharmaceutical products, dietary supplements and cosmetic formulations. Non-limiting examples of such products are tablets, capsules, ointments and lotions as described above.

### Examples 1 to 19

### Molar Ratio Pyridine Carboxy Derivative/Aminosugar Derivative 1:10000 (mol/mol).

| | Pyridine carboxy | |
|---|---|---|
| | derivative (1 mol) | Aminosugar (10000 mol) |
| Example 1. | Niacinamide | Glucosamine |
| Example 2. | Niacinamide | Glucosamine HCl |
| Example 3. | Niacinamide | Glucosamine potassium sulfate salt |
| Example 4. | Niacinamide | Glucosamine 2 sulfate, free acid |
| Example 5. | Niacinamide | Glucosamine 2 sulfate, Na⁺ salt |
| Example 6. | Thioniacinamide | Glucosamine 3 sulfate, free acid |
| Example 7. | Niacinamide | Glucosamine 3 sulfate, K⁺ salt |
| Example 8. | Niacinamide | N-acetylglucosamine 3,6 sulfate, di |
| | | Na⁺ salt |
| Example 9. | Niacinamide | N-acetylglucosamine 3,4,6 sulfate, |
| | | Na⁺ salt |
| Example 10. | Niacinamide | N-acetylglucosamine 3,4,6 sulfate, |
| | | tri Na⁺ salt |
| Example 11. | Niacinamide | Galactosamine 3,6 sulfate, di K⁺ |
| | | salt |
| Example 12. | Niacinamide | Galactosamine 3,4,6 sulfate, di Na⁺ |
| | | salt |
| Example 13. | Aminoniacinamide | N-acetylgalactosamine |
| Example 14. | Niacinamide | N-acetylgalactosamine 3 sulfate, |
| | | Na⁺ salt |
| Example 15. | Niacinamide | N-acetylgalactosamine 3 sulfate, |
| | | K⁺ salt |
| Example 16. | N2-methyl- | Glucosamine |
| | niacinamide | |
| Example 17. | N2-methyl- | Glucosamine HCl |
| | niacinamide | |
| Example 18. | N2-ethyl-niacinamide | Galactosamine 3,4,6 sulfate, di Na⁺ |
| | | salt |
| Example 19. | N2-ethyl-niacinamide | N-acetylgalactosamine |

### Examples 20 to 34

### Molar Ratio Pyridine Carboxy Derivative/Aminosugar Derivative 1:1000 (mol/mol).

| | pyridine carboxy | |
|---|---|---|
| | derivative (1 mol) | Aminosugar (1000 mol) |
| Example 20. | Niacinamide | Glucosamine |
| Example 21. | Niacinamide | Glucosamine HCl |
| Example 22. | Niacinamide | Glucosamine sodium sulfate salt |
| Example 23. | Thioniacinamide | Galactosamine |
| Example 24. | Niacinamide | Galactosamine HCl |
| Example 25. | Niacinamide | Galactosamine potassium sulfate |
| | | salt |
| Example 26. | Niacinamide | N-acetylgalactosamine 6 sulfate, |
| | | Na⁺ salt |
| Example 27. | Aminoniacinamide | N-acetylgalactosamine 6 sulfate, |
| | | K⁺ salt |
| Example 28. | Niacinamide | N-acetylgalactosamine 3,6 sulfate, |
| | | free acid |
| Example 29. | Niacinamide | N-acetylgalactosamine 3,6 sulfate, |
| | | Na⁺ salt |
| Example 30. | N2-methyl- | N-acetylgalactosamine |
| | niacinamide | |
| Example 31. | N2-methyl- | N-acetylgalactosamine potassium |
| | niacinamide | sulfate salt |
| Example 32. | N2-ethyl-niacinamide | Glucosamine 2 sulfate, Na⁺ salt |
| Example 33. | N2-ethyl-niacinamide | Glucosamine 3 sulfate, free acid |
| Example 34. | N2-ethyl-niacinamide | Glucosamine 3 sulfate, K⁺ salt |

### Examples 35 to 55

### Molar Ratio Pyridine Carboxy Derivative/Aminosugar Derivative 1:100 (mol/mol).

| | pyridine carboxy | |
|---|---|---|
| | derivative (1 mol) | Aminosugar (100 mol) |
| Example 35. | Niacinamide | Glucosamine |
| Example 36. | Niacinamide | Glucosamine HCl |
| Example 37. | Niacinamide | Glucosamine potassium sulfate salt |
| Example 38. | Thioniacinamide | Glucosamine 2 sulfate, free acid |
| Example 39. | Niacinamide | Glucosamine 3 sulfate, K⁺ salt |
| Example 40. | Niacinamide | Glucosamine 6 sulfate, Na⁺ salt |
| Example 41. | Niacinamide | Glucosamine 2,3 sulfate, free acid |
| Example 42. | Niacinamide | Glucosamine 2,3 sulfate, di Na⁺ |
| | | salt |
| Example 43. | Aminoniacinamide | N-acetylglucosamine HCl |
| Example 44. | Niacinamide | N-acetylglucosamine 3 sulfate, Na⁺ |
| | | salt |
| Example 45. | Niacinamide | Galactosamine 3,6 sulfate, K⁺ salt |
| Example 46. | Niacinamide | Galactosamine 3,4,6 sulfate, di Na⁺ |
| | | salt |
| Example 47. | Niacinamide | Galactosamine 3,4,6 sulfate, tri |
| | | Na⁺ salt |
| Example 48. | Niacinamide | N-acetylgalactosamine |
| Example 49. | Niacinamide | N-acetylgalactosamine sodium |
| | | sulfate salt |
| Example 50. | Niacinamide | N-acetylgalactosamine HCl |
| Example 51. | N2-ethyl-niacinamide | N-acetylgalactosamine |
| Example 52. | N2-ethyl-niacinamide | N-acetylgalactosamine 3 sulfate, |
| | | Na⁺ salt |
| Example 53. | N2-ethyl-niacinamide | N-acetylgalactosamine 3 sulfate, |
| | | K⁺ salt |
| Example 54. | N2-methyl- | Glucosamine HCl |
| | niacinamide | |
| Example 55. | N2-methyl- | Glucosamine potassium sulfate salt |
| | niacinamide | |

### Examples 56 to 65

### Molar Ratio Pyridine Carboxy Derivative/Pyridine Carboxy Derivative 1:50 (mol/mol).

| | pyridine carboxy | |
|---|---|---|
| | derivative (1 mol) | Aminosugar (50 mol) |
| Example 56. | Niacinamide | Glucosamine |
| Example 57. | Niacinamide | Glucosamine HCl |
| Example 58. | Niacinamide | Glucosamine potassium sulfate salt |
| Example 59. | Thioniacinamide | Glucosamine 2 sulfate, free acid |
| Example 60. | Niacinamide | Glucosamine 2 sulfate, Na⁺ salt |
| Example 61. | Niacinamide | N-acetylgalactosamine 3 sulfate, free |
| | | acid |
| Example 62. | Niacinamide | N-acetylgalactosamine 3 sulfate, Na⁺ |
| | | salt |
| Example 63. | Aminoniacinamide | N-acetylgalactosamine 4 sulfate, K⁺ |
| | | salt |
| Example 64. | Niacinamide | N-acetylgalactosamine 6 sulfate, free |
| | | acid |
| Example 65. | Niacinamide | N-acetylgalactosamine 3,6 sulfate, |
| | | Na⁺ salt |

### Examples 66 to 74

### Molar Ratio Pyridine Carboxy Derivative/Aminosugar Derivative 1:2 (mol/mol).

| | pyridine carboxy | |
|---|---|---|
| | derivative (1 mol) | Aminosugar (2 mol) |
| Example 66. | Niacinamide | N-acetylgalactosamine 3,6 sulfate, |
| | | di Na⁺ salt |
| Example 67. | Niacinamide | N-acetylgalactosamine 3,6 sulfate, |
| | | K⁺ salt |
| Example 68. | Niacinamide | N-acetylglucosamin |
| Example 69. | Niacinamide | N-acetylgalactosamine 3,4,6 |
| | | sulfate, Na⁺ salt |
| Example 70. | Niacinamide | N-acetylgalactosamine 3,4,6 |
| | | sulfate, di Na⁺ salt |
| Example 71. | Niacinamide | N-acetylgalactosamine 3,4,6 |
| | | sulfate, tri Na⁺ salt |
| Example 72. | N2-methyl- | Galactosamine HCl |
| | niacinamide | |
| Example 73. | N2-methyl- | Galactosamine potassium sulfate |
| | niacinamide | salt |
| Example 74. | N2-ethyl-niacinamide | N-acetylgalactosamine 6 sulfate, |
| | | Na⁺ salt |

### Examples 75 to 91

### Molar Ratio Pyridine Carboxy Derivative/Aminosugar Derivative 2:3 (mol/mol).

| | Example 76. | |
|---|---|---|
| | pyridine carboxy | Example 77. |
| Example 75. | derivative (2 mol) | Aminosugar (3 mol) |
| Example 78. | Niacinamide | Glucosamine |
| Example 79. | Thioniacinamide | Glucosamine HCl |
| Example 80. | Niacinamide | Glucosamine potassium sulfate salt |
| Example 81. | N2-ethyl-niacinamide | Glucosamine 2 sulfate, free acid |
| Example 82. | Niacinamide | Glucosamine 3 sulfate, Na+ salt |
| Example 83. | Niacinamide | Glucosamine 6 sulfate, K+ salt |
| Example 84. | Aminoniacinamide | Glucosamine 2,3 sulfate, di Na+ |
| | | salt |
| Example 85. | Niacinamide | Glucosamine 2,6 sulfate, Na+ salt |
| Example 86. | Niacinamide | Glucosamine 3,4,6 sulfate, free |
| | | acid |
| Example 87. | Niacinamide | N-acetylglucosamine |
| Example 88. | Niacinamide | N-acetylglucosamine HCl |
| Example 89. | N2-ethyl-niacinamide | N-acetylglucosamine 3 sulfate, |
| | | Na⁺ salt |
| Example 90. | Thioniacinamide | N-acetylglucosamine 6 sulfate, |
| | | Na⁺ salt |
| Example 91. | Aminoniacinamide | N-acetylglucosamine 3,4,6 sulfate, |
| | | tri Na⁺ salt |
| Example 92. | Niacinamide | Galactosamine |
| Example 93. | N2-methyl- | Galactosamine HCl |
| | niacinamide | |
| Example 94. | Niacinamide | Galactosamine sodium sulfate salt |

### Examples 93 to 116

### Molar Ratio Pyridine Carboxy Derivative/Aminosugar Derivative 1:1 (mol/mol)

| | pyridine carboxy | |
|---|---|---|
| | derivative (1 mol) | Aminosugar (1 mol) |
| Example 95. | Niacinamide | Glucosamine |
| Example 96. | Thioniacinamide | Glucosamine HCl |
| Example 97. | Niacinamide | Glucosamine potassium sulfate salt |
| Example 98. | Aminoniacinamide | Glucosamine 2,3 sulfate, di Na⁺ |
| | | salt |
| Example 99. | Niacinamide | Glucosamine 3,4,6 sulfate, free |
| | | acid |
| Example 100. | Niacinamide | N-acetylglucosamine |
| Example 101. | Niacinamide | N-acetylglucosamine HCl |
| Example 102. | N2-ethyl- | N-acetylglucosamine 3 sulfate, Na⁺ |
| | niacinamide | salt |
| Example 103. | Thioniacinamide | N-acetylglucosamine 6 sulfate, Na⁺ |
| | | salt |
| Example 104. | Niacinamide | N-acetylglucosamine 6 sulfate, |
| | | K⁺ salt |
| Example 105. | N2-methyl- | N-acetylglucosamine 3,6 sulfate, |
| | niacinamide | di Na⁺ salt |
| Example 106. | Niacinamide | N-acetylglucosamine 3,4,6 sulfate, |
| | | Na⁺ salt |
| Example 107. | Aminoniacinamide | N-acetylglucosamine 3,4,6 sulfate, |
| | | tri Na⁺ salt |
| Example 108. | Niacinamide | Galactosamine |
| Example 109. | N2-methyl- | Galactosamine HCl |
| | niacinamide | |
| Example 110. | Niacinamide | Galactosamine sodium sulfate salt |
| Example 111. | Niacinamide | Galactosamine 3 sulfate, K⁺ salt |
| Example 112. | Thioniacinamide | Galactosamine 4 sulfate, Na⁺ salt |
| Example 113. | Niacinamide | Galactosamine 6 sulfate, K⁺ salt |
| Example 114. | Niacinamide | Galactosamine 2,3 sulfate, di Na⁺ |
| | | salt |
| Example 115. | Aminoniacinamide | Galactosamine 2,3 sulfate, K⁺ |
| | | salt |
| Example 116. | Niacinamide | N-acetylgalactosamine 4 sulfate, |
| | | K⁺ salt |
| Example 117. | N2-methyl- | N-acetylgalactosamine 6 sulfate, |
| | niacinamide | free acid |
| Example 118. | Niacinamide | N-acetylgalactosamine 3,6 sulfate, |
| | | di Na⁺ salt |
| Example 119. | Niacinamide | N-acetylgalactosamine 3,4,6 |
| | | sulfate, K⁺ salt |

### Examples 120 to 133

### Molar Ratio Pyridine Carboxy Derivative/Aminosugar Derivative 2:1 (mol/mol).

| | pyridine carboxy | |
|---|---|---|
| | derivative (2 mol) | Aminosugar (1 mol) |
| Example 120. | Niacinamide | Glucosamine 2,3 sulfate, free acid |
| Example 121. | Niacinamide | Glucosamine 2,3 sulfate, di Na⁺ |
| | | salt |
| Example 122. | Niacinamide | Glucosamine 2,6 sulfate, Na⁺ salt |
| Example 123. | thioniacinamide | Glucosamine 3,6 sulfate, di Na⁺ |
| | | salt |
| Example 124. | Niacinamide | Glucosamine 3,4,6 sulfate, free |
| | | acid |
| Example 125. | Niacinamide | N-acetylglucosamine |
| Example 126. | N2-methyl- | N-acetylglucosamine HCl |
| | niacinamide | |
| Example 127. | N2-ethyl- | N-acetylglucosamine 3 sulfate, free |
| | niacinamide | acid |
| Example 128. | N2-methyl- | N-acetylglucosamine 3 sulfate, Na⁺ |
| | niacinamide | salt |
| Example 129. | N2-ethyl- | Galactosamine 3,4,6 sulfate, di Na⁺ |
| | niacinamide | salt |
| Example 130. | Niacinamide | Galactosamine 3,4,6 sulfate, tri |
| | | Na⁺ salt |
| Example 131. | Thioniacinamide | N-acetylgalactosamine |
| Example 132. | Niacinamide | N-acetylgalactosamine potassium |
| | | sulfate salt |
| Example 133. | Niacinamide | N-acetylgalactosamine HCl |

### Example 133

### Niacinamide N-acetylgalactosamine HCl

| | pyridine carboxy | |
|---|---|---|
| | derivative (5 mol) | Aminosugar (1 mol) |
| Example 134. | Niacinamide | Glucosamine |
| Example 135. | Niacinamide | Glucosamine HCl |
| Example 136. | Niacinamide | Glucosamine potassium sulfate salt |
| Example 137. | N2-ethyl- | Glucosamine 2 sulfate, free acid |
| | niacinamide | |
| Example 138. | Niacinamide | Glucosamine 3 sulfate, Na⁺ salt |
| Example 139. | Niacinamide | Glucosamine 6 sulfate, K⁺ salt |
| Example 140. | Niacinamide | Glucosamine 2,3 sulfate, di Na⁺ salt |
| Example 141. | Niacinamide | Glucosamine 2,6 sulfate, Na⁺ salt |
| Example 142. | Niacinamide | Glucosamine 3,4,6 sulfate, free acid |

### Examples 140 to 157

### Molar Ratio Pyridine Carboxy Derivative/Aminosugar Derivative 50:1 (mol/mol).

| | pyridine carboxy | |
|---|---|---|
| | derivative (50 mol) | Aminosugar (1 mol) |
| Example 143. | Niacinamide | Glucosamine |
| Example 144. | Thioniacinamide | Glucosamine HCl |
| Example 145. | Niacinamide | Glucosamine sodium sulfate salt |
| Example 146. | Niacinamide | Glucosamine 2 sulfate, Na⁺ salt |
| Example 147. | Niacinamide | N-acetylglucosamine 3,6 sulfate, di |
| | | Na⁺ salt |
| Example 148. | Niacinamide | N-acetylglucosamine 3,4,6 sulfate, |
| | | di K⁺ salt |
| Example 149. | Niacinamide | Galactosamine 2 sulfate, Na⁺ salt |
| Example 150. | Aminoniacin- | Galactosamine 2 sulfate, K⁺ salt |
| | amide | |
| Example 151. | Niacinamide | Galactosamine 3 sulfate, free acid |
| Example 152. | Niacinamide | N-acetylgalactosamine 3 sulfate, |
| | | K⁺ salt |
| Example 153. | Niacinamide | N-acetylgalactosamine 4 sulfate, |
| | | K⁺ salt |
| Example 154. | Thioniacinamide | N-acetylgalactosamine 6 sulfate, |
| | | Na⁺ salt |
| Example 155. | N2-methyl- | N-acetylgalactosamine 6 sulfate, |
| | niacinamide | K⁺ salt |
| Example 156. | N2-ethyl- | N-acetylgalactosamine 3,6 sulfate, |
| | niacinamide | free acid |
| Example 157. | N2-methyl- | N-acetylgalactosamine 3,6 sulfate, |
| | niacinamide | Na⁺ salt |
| Example 158. | N2-ethyl- | N-acetylgalactosamine 3,6 sulfate, |
| | niacinamide | di Na⁺ salt |
| Example 159. | N2-methyl- | N-acetylgalactosamine 3,4,6 |
| | niacinamide | sulfate, di Na⁺ salt |
| Example 160. | N2-ethyl- | N-acetylgalactosamine 3,4,6 |
| | niacinamide | sulfate, tri Na⁺ salt |

### Examples 161 to 177

### Molar Ratio Pyridine Carboxy Derivative/Aminosugar Derivative 500:1 (mol/mol).

| | pyridine carboxy | |
|---|---|---|
| | derivative (500 mol) | Aminosugar (1 mol) |
| Example 161. | Niacinamide | Glucosamine |
| Example 162. | Niacinamide | Glucosamine HCl |
| Example 163. | Thioniacinamide | Glucosamine potassium sulfate salt |
| Example 164. | Niacinamide | Glucosamine 2 sulfate, free acid |
| Example 165. | Niacinamide | Glucosamine 2 sulfate, Na⁺ salt |
| Example 166. | Niacinamide | Glucosamine 2 sulfate, K⁺ salt |
| Example 167. | Niacinamide | Glucosamine 3 sulfate, free acid |
| Example 168. | Aminoniacinamide | Glucosamine 3 sulfate, Na⁺ salt |
| Example 169. | Niacinamide | Glucosamine 6 sulfate, free acid |
| Example 170. | Niacinamide | Glucosamine 6 sulfate, Na⁺ salt |
| Example 171. | Niacinamide | N-acetylglucosamine 3,6 sulfate, di |
| | | Na⁺ salt |
| Example 172. | Niacinamide | N-acetylglucosamine 3,4,6 sulfate, |
| | | di K⁺ salt |
| Example 173. | Niacinamide | N-acetylglucosamine 3,4,6 sulfate, |
| | | Na⁺ salt |
| Example 174. | N2-methyl- | N-acetylglucosamine 3,4,6 sulfate, |
| | niacinamide | di Na⁺ salt |
| Example 175. | N2-ethyl- | N-acetylglucosamine 3,4,6 sulfate, |
| | niacinamide | tri Na⁺ salt |
| Example 176. | N2-methyl- | Galactosamine 3,6 sulfate, K⁺ |
| | niacinamide | salt |
| Example 177. | N2-ethyl- | Galactosamine 3,6 sulfate, di K⁺ |
| | niacinamide | salt |
| Example 178. | N2-methyl- | Galactosamine 3,4,6 sulfate, di Na⁺ |
| | niacinamide | salt |
| Example 179. | N2-ethyl- | Galactosamine 3,4,6 sulfate, tri |
| | niacinamide | Na⁺ salt |
| Example 180. | N2-methyl- | N-acetylgalactosamine |
| | niacinamide | |

### Examples 181 to 190

### Molar Ratio Pyridine Carboxy Derivative/Aminosugar Derivative 5000:1 (mol/mol).

| | pyridine carboxy | |
|---|---|---|
| | derivative (5000 mol) | Aminosugar (1 mol) |
| Example 181. | Niacinamide | Glucosamine |
| Example 182. | Niacinamide | Glucosamine HCl |
| Example 183. | Niacinamide | Glucosamine sodium sulfate salt |
| Example 184. | Thioniacinamide | Galactosamine |
| Example 185. | Niacinamide | Galactosamine HCl |
| Example 186. | Niacinamide | Galactosamine potassium sulfate |
| | | salt |
| Example 187. | N2-ethyl- | N-acetylgalactosamine 6 sulfate, |
| | niacinamide | Na⁺ salt |
| Example 188. | N2-methyl- | N-acetylgalactosamine 6 sulfate, |
| | niacinamide | K⁺ salt |
| Example 189. | N2-ethyl- | N-acetylgalactosamine 3,6 |
| | niacinamide | sulfate, free acid |
| Example 190. | N2-methyl- | N-acetylgalactosamine 3,6 |
| | niacinamide | sulfate, Na⁺ salt |

### Examples 190 to 200

### Molar Ratio Pyridine Carboxy Derivative/Aminosugar Derivative 10000:1 (mol/mol).

| | pyridine carboxy | |
|---|---|---|
| | derivative | |
| | (10000 mol) | Aminosugar (1 mol) |
| Example 191. | Niacinamide | Glucosamine 2,3 sulfate, di Na⁺ |
| | | salt |
| Example 192. | Thioniacinamide | Glucosamine 2,6 sulfate, Na⁺ |
| | | salt |
| Example 193. | Niacinamide | Glucosamine 3,6 sulfate, di Na⁺ |
| | | salt |
| Example 194. | Niacinamide | Glucosamine 3,4,6 sulfate, free |
| | | acid |
| Example 195. | Aminoniacinamide | N-acetylglucosamine |
| Example 196. | Niacinamide | N-acetylglucosamine HCl |
| Example 197. | Niacinamide | N-acetylglucosamine 3 sulfate, free |
| | | acid |
| Example 198. | Niacinamide | N-acetylglucosamine 3 sulfate, Na⁺ |
| | | salt |
| Example 199. | Niacinamide | N-acetylglucosamine 6 sulfate, Na⁺ |
| | | salt |
| Example 200. | N2-ethyl- | N-acetylglucosamine 6 sulfate, K⁺ |
| | niacinamide | salt |
| Example 201. | N2-methyl- | Galactosamine HCl |
| | niacinamide | |
| Example 202. | N2-ethyl- | Galactosamine potassium sulfate |
| | niacinamide | salt |
| Example 203. | N2-methyl- | N-acetylgalactosamine 6 sulfate, |
| | niacinamide | K⁺ salt |

### Examples 201 to 216

### Weight Ratio Pyridine Carboxy Derivative/Aminosugar Derivative 1:1 (g/g).

| | pyridine carboxy | |
|---|---|---|
| | derivative (1000 g) | Aminosugar (1000 g) |
| Example 204. | Niacinamide | Glucosamine |
| Example 205. | Thioniacinamide | Glucosamine HCl |
| Example 206. | Niacinamide | Glucosamine potassium sulfate salt |
| Example 207. | Niacinamide | Glucosamine 2 sulfate, free acid |
| Example 208. | Niacinamide | Glucosamine 2 sulfate, Na⁺ salt |
| Example 209. | Niacinamide | Glucosamine 2 sulfate, K⁺ salt |
| Example 210. | Aminoniacinamide | Galactosamine |
| Example 211. | Niacinamide | Galactosamine HCl |
| Example 212. | Niacinamide | Galactosamine potassium sulfate |
| | | salt |
| Example 213. | Niacinamide | Galactosamine 2 sulfate, free acid |
| Example 214. | Niacinamide | Galactosamine 2 sulfate, Na⁺ salt |
| Example 215. | Thioniacinamide | Galactosamine 2 sulfate, K⁺ salt |
| Example 216. | Niacinamide | N-acetylgalactosamine |
| Example 217. | Niacinamide | N-acetylgalactosamine sodium |
| | | sulfate salt |
| Example 218. | N2-ethyl- | N-acetylgalactosamine HCl |
| | niacinamide | |
| Example 219. | N2-methyl- | N-acetylgalactosamine 3 sulfate, |
| | niacinamide | free acid |

### Examples 218 to 231

### Weight Ratio Pyridine Carboxy Derivative/Aminosugar Derivative 10:1 (g/g).

| | pyridine carboxy | |
|---|---|---|
| | derivative (1000 g) | Aminosugar (100 g) |
| Example 220. | Niacinamide | N-acetylgalactosamine 4 sulfate, |
| | | K⁺ salt |
| Example 221. | Niacinamide | N-acetylgalactosamine 6 sulfate, |
| | | Na⁺ salt |
| Example 222. | Niacinamide | N-acetylgalactosamine 6 sulfate, |
| | | K⁺ salt |
| Example 223. | Thioniacinamide | N-acetylgalactosamine 3,6 sulfate, |
| | | Na⁺ salt |
| Example 224. | Niacinamide | N-acetylgalactosamine 3,6 sulfate, |
| | | di Na⁺ salt |
| Example 225. | Niacinamide | Glucosamine 2,6 sulfate, Na⁺ salt |
| Example 226. | Niacinamide | Glucosamine 3,6 sulfate, di Na⁺ |
| | | salt |
| Example 227. | Niacinamide | Glucosamine 3,4,6 sulfate, free |
| | | acid |
| Example 228. | Aminoniacinamide | N-acetylglucosamine |
| Example 229. | Niacinamide | N-acetylglucosamine HCl |
| Example 230. | Niacinamide | N-acetylglucosamine 3 sulfate, free |
| | | acid |
| Example 231. | N2-ethyl- | N-acetylglucosamine 3 sulfate, Na⁺ |
| | niacinamide | salt |
| Example 232. | N2-methyl- | N-acetylglucosamine 6 sulfate, Na⁺ |
| | niacinamide | salt |
| Example 233. | N2-ethyl- | N-acetylglucosamine 6 sulfate, Ka⁺ |
| | niacinamide | salt |
| Example 234. | N2-methyl- | N-acetylglucosamine 3,6 sulfate, di |
| | niacinamide | Na⁺ salt |

### General Method Examples 232-238:

Pharmaceutical compositions according to the invention are prepared. A quantity of the pyridine carboxy derivative and the aminosugar derivative are transferred to a hard gelatine capsule.

### Examples 232 to 235

### Capsule 500 mg, Molar Ratio Pyridine Carboxy Derivative/Aminosugar Derivate 5:1

| | pyridine carboxy | |
|---|---|---|
| | derivative quantity | Aminosugar quantity |
| Example 235. | Niacinamide 250 g | Glucosamine potassium sulfate |
| | | salt 250 g |
| Example 236. | Niacinamide 367 g | N-acetylglucosamine 133 g |
| Example 237. | Niacinamide 370 g | Galactosamine HCl 130 g |
| Example 238. | Niacinamide 342 g | Glucosamine 2 sulfate, Na⁺ salt |
| | | 158 g |

### Examples 236 to 238

### Capsule 250 mg, Molar Ratio Pyridine Carboxy Derivative/Aminosugar Derivate 7:4

| | pyridine carboxy | |
|---|---|---|
| | derivative quantity | Aminosugar quantity |
| Example 239. | Niacinamide 65 g | Glucosamine potassium sulfate salt |
| | | 185 g |
| Example 240. | Niacinamide 123 g | N-acetylglucosamine 127 g |
| Example 241. | Niacinamide 124 g | Galactosamine HCl 126 g |

### Example 239

Objective

The objective of this study is to assess the effect of two doses of two chemical complexes of the invention topically administered in the tetradecanoyl phorbol acetate (TPA) induced ear inflammation test in the mouse, a commonly employed method for screening and evaluation of antiinflammatory drugs. Locoid® cutaneous solution (0.1% hydrocortisone 17-butyrate) is used as a positive control.

Test Articles and Vehicle

The test articles are the complexes of the invention prepared according to example 133 and example 122 (Compound 133 and Compound 122 in the following). Compound 133, Compound 122 and Locoid® cutaneous solution (hydrocortisone 17-butyrate) are obtained from Astion A/S, Denmark.

Animals

The study is performed in 63 female SPF NMRI mice of the stock Bom:NMRI from M & B A/S, DK-8680 Ry. At start of the acclimatisation period the mice is in the weight range of 18-20 g. An acclimatisation period of 7 days is allowed.

Housing

The study will take place in an animal room provided with filtered air. The temperature in the room is set at 21-23° C. and the relative humidity to ≧50%. The room is illuminated to give a cycle of 12 hours light and 12 hours darkness. Light is on from 06.00 till 18.00 h. The animals is housed in Macrolon type III cages (40×25×14 cm), nine in each cage. The cages is cleaned and the bedding changed at least once a week. The animal room is cleaned and disinfected with Diversol Bx. The animals will have free access to bottles with domestic quality drinking water added citric acid to pH 3.

Animal Randomisation and Allocation

On the day of arrival the animals is randomly allocated to 7 groups, each of 9 mice.

Animal and Cage Identification

Each animal is identified by coloured marks on the tails. Each cage is marked with study number 2021, cage number, group number and animal numbers.

Procedure

The test substances are applied in 20 μl volumes to the inner surface of the right ear on day 0. 20 minutes before and again 20 minutes after TPA treatment. All groups is treated with 20 μl acetone on the left ear and with 20 μl TPA, 400 μg/ml, on the right ear.

The groups, doses and animal numbers is as follows:

| | | Dose, mg per | |
|---|---|---|---|
| Group | Drug, left/right ear | application | Animal numbers |
| 1 | −/− | — | 1-9 |
| 2 | −/Vehicle | — | 10-18 |
| 3 | compound 133 | 3.5 | 19-27 |
| 4 | compound 133 | 7.0 | 28-36 |
| 5 | compound 122 | 3.5 | 37-45 |
| 6 | compound 122 | 7.0 | 46-54 |
| 7 | −/Locoid solution, 0.1% | 0.02 | 55-63 |

Three hours after the TPA application the mice are sacrificed, the ears cut off and weighed. Mean weights and standard deviations are calculated. Percent inhibition of the oedema compared with group 1 is calculated for the groups 2-7. Weights of the left ears is used to assess the comparability of the groups and to calculate differences in weight between left and right ear.

Findings

Ear swelling is determined as the difference between the weight of right and left ear. Compound 133 gave an inhibition of ear swelling of 55 and 78% at 3.5 mg/ear and 7 mg/ear, respectively. Compound 122 gave an inhibition of ear swelling of 73 and 98% at 3.5 mg/ear and 7 mg/ear, respectively. Hydrocortisone 17-butyrate solution gave an inhibition of ear swelling of 90%.

Conclusion

Compound 133 and Compound 122 inhibited ear swelling dose-dependently and at a level comparable to hydrocortisone 17-butyrate.

### Example 240

In a small preliminary clinical investigation eight persons administered a topical pharmaceutical composition according to the invention. The composition was a cream according to the following formula:

| | | |
|---|---|---|
| Water, purified | 57.9% | (w/w) |
| Tefose 63 (Gattefosse) | 12% | (w/w) |
| Vaselin Ph. Eur. | 10% | (w/w) |
| Paraffin oil Ph. Eur. | 10% | (W/W) |
| Compound 122* | 10% | (w/w) |
| Methyl parabene | 0.1% | (w/w) |

*Compound 122 is the complex of the invention prepared in example 122.

One patient (female) was 35 years old and had suffered from nodular prurigo (prurigo nodularis Hyde) for 18 years. The patient had previously for periods been treated with strong topical steroids without significant effect on her inflamed nodules and strong (almost unbearable) itch. She had also for a period been treated with systemic thalidomide, but with limited effect and unacceptable adverse effects. Therefore she had been without treatment for the last couple of years and the disease was characterised by inflamed nodules and strong itch. After applying the cream according to the invention for the first time she observed a significant decrease of her symptoms. The cream was applied twice daily and was able to completely remove her symptoms. She reported having been able to sleep at night without problems for the first time in 18 years. The treatment continued for 6 months with the same consistent result. Several attempts of not using the cream led to dramatic reoccurrence of symptoms within two days and reapplication of the cream could every time lead to complete absence of symptoms.

Another patient (female) was 55 years old had suffered from contact dermatitis for three years. The dermatitis was located on the hands and the neck and characterised by erythema. The patient had for periods been treated with strong topical steroids with no or limited effect. The cream according to the invention was applied twice daily. After two weeks there was a significant improvement, which was maintained for the entire treatment period of 5 months.

Another patient (female) was 85 years old and suffered from senile pruritus with strong symptoms on arms and legs. She was hospitalised for two weeks and treated with strong topical steroids and antipsychotics with very limited result. The cream according to the invention was then applied twice daily and a marked improvement was observed within two days. After two weeks of treatment the patient was further improved to an extent where hospitalisation was no longer required.

Another patient (female) was 35 years old and suffered from keloids on the arms and the chest. The keloids were especially sore and itching in the armpits. The cream according to the invention was applied twice daily. The symptoms of soreness and itching from the keloids dissapeared within a couple of hours after the first application of the cream. The alleviation of symptoms was maintained with the twice-daily application.

Another patient (female) was 50 years old and had suffered from psoriasis located on the elbows, knees and legs for more than 20 years. The cream according to the invention was applied twice daily for 5 months. Over the entire period a gradual and significant improvement was observed, which was significantly better than for untreated control elements.

Another patient (male) was 26 years old and had suffered from seborrhoeic dermatitis on both sides of the nose for five years. The dermatitis was characterised by strong erythema and some scaling. The cream according to the invention was applied twice daily. A clear improvement of erythema and scaling was observed within a week and after 14 days the dermatitis was completely gone and the treatment was terminated. The following 7 months the patient used the cream occasionally when the symptoms reappeared and the symptoms disappeared completely every time after two to three days treatment.

Another patient (male) was 55 years old and had suffered from seborrhoeic dermatitis all over the face and on the scalp for 10 years. The dermatitis was characterised by strong erythema, soreness and scaling, especially on the scalp. The cream according to the invention was applied twice daily and a gradual improvement was observed over two weeks. After three weeks the facial symptoms had completely gone and the scalp symptoms had improved significantly.

Another patient (male) was 58 years old and had suffered from seborrhoeic dermatitis of the chest and scalp for 30 years. The dermatitis was especially characterised by scaling and itching. The cream according to the invention was applied twice daily to the chest and the dermatitis was completely gone after 10 days of treatment. Every 6 weeks it was necessary to reapply the cream due to reoccurrence of the dermatitis for two to three days, which completely removed the symptoms. The subject used a standard shampoo added compound 122 to a final concentration of 10% (w/w) on the scalp. This shampoo improved the scalp dermatitis gradually and within two weeks the dermatitis was reduced significantly.

### Example 241

Objective

The objective of this study is to assess the effect of Compound 122 as compared to its components in the tetradecanoyl phorbol acetate (TPA) induced ear inflammation test in the mouse, a commonly employed method for screening and evaluation of antiinflammatory drugs. Locoid® cutaneous solution (0.1% hydrocortisone 17-butyrate) is used as a positive control.

Test Articles and Vehicle

The test articles are the complex of the invention prepared according to example 118 (Compound 122 in the following) and its components niacinamide and N-acetylglucosamine, all obtained from Astion A/S, Denmark.

Animals

The study is performed in female SPF NMRI mice of the stock Bom:NMRI from M & B A/S, Denmark. At start of the acclimatisation period the mice is in the weight range of 18-20 g. An acclimatisation period of 7 days is allowed.

Housing

The study takes place in an animal room provided with filtered air. The temperature in the room is set at 21-23° C. and the relative humidity to >50%. The room is illuminated to give a cycle of 12 hours light and 12 hours darkness. Light is on from 06.00 till 18.00 h.

The animals are housed in Macrolon type III cages (40×25×14 cm), nine in each cage. The cages are cleaned and the bedding changed at least once a week. The animal room is cleaned and disinfected with Diversol Bx.

Procedure

On day 0 the test substances are applied in 20 μl volumes to the inner surface of the right ear 20 minutes before and again 20 minutes after TPA treatment. All groups are treated with 20 μl acetone on the left ear and with 20 μl TPA, 400 μg/ml, on the right ear.

Three hours after the TPA application the mice are sacrificed, the ears cut off and weighed.

The following groups are included in the study:

| | | Dose, mg per |
|---|---|---|
| Group | Drug | application |
| A | Vehicle control | — |
| B | Compound 122 | 7.0 |
| C | Niacinamide | 3.67 |
| D | N-acetylglucosamine | 3.33 |
| E | Hydrocortisone 17-butyrate | 0.02 |

The amount of niacinamide and N-acetylglucosamine administered to groups C and D corresponds exactly to the amount of the two substances present in Compound 122 administered to group B.

Mean weights and standard deviations are calculated. Percent inhibition of the oedema compared with group A is calculated for the groups B-E. Weights of the left ears are used to assess the comparability of the groups and to calculate differences in weight between left and right ear.

Findings

Ear swelling is determined as the difference between the weight of right and left ear. The following inhibition of ear swelling is observed in groups B-E:

| | | Dose, mg per | Inhibition of ear |
|---|---|---|---|
| Group | Drug | application | swelling (%) |
| B | Compound 122 | 7.0 | 79*** |
| C | Niacinamide | 3.67 | 31** |
| D | N-acetylglucosamine | 3.33 | 31* |
| E | Hydrocortisone 17-butyrate | 0.02 | 89*** |

***p < 0.001 (Mann-Whitney U test)
**p < 0.01 (Mann-Whitney U test)
*p < 0.05 (Mann-Whitney U test)

Compound 122 inhibited ear swelling significantly and in the same order of magnitude as the positive control hydrocortisone 17-butyrate, while niacinamide and N-acetylglucosamine only displayed a modest inhibition.

The inhibition obtained with Compound 122 (group B) is 27% higher than the theoretical additive inhibition of the components of Compound 122 (group C+group D) thus displaying a synergistic effect.

Conclusion

Compound 122 inhibited ear swelling synergistically as compared to its components and in the same order of magnitude as a clinically relevant dose of hydrocortisone 17-butyrate.

1. A chemical complex consisting of:
i) one or more optionally substituted pyridine carboxy derivative(s) or salt(s) thereof according to formula I 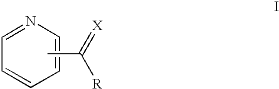
wherein X is selected from O and S; R is selected from OH; OR′; NH₂; NHR′; NR′R″, O⁻Y⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and
ii) one or more optionally substituted aminosugar(s) or salt(s) thereof,
wherein the one or more optionally substituted aminosugar(s) is/are aminosugar derivative(s) of a mono-saccharide or an oligo-saccharide containing of at the most of six saccharide units.

2. The chemical complex according to claim 1, wherein the one or more optionally substituted aminosugar(s) is/are aminosugar derivative(s) of a mono-saccharide or a di-saccharide.

3. The chemical complex according to claim 1, the one or more optionally substituted aminosugar(s) is/are aminosugar derivative(s) of a mono-saccharide.

4. The chemical complex according to claim 3, wherein said aminosugar derivative of a mono-saccharide is selected from the group consisting of glucosamine, galactosamine or mannosamine, their derivatives and salts thereof.

5. The chemical complex according to claim 4, wherein said aminosugar derivative of a mono-saccharide is selected from the group consisting of glucosamine sulfate, glucosamine hydrochloride, N-acetylglucosamine, galactosamine sulfate, galactosamine hydrochloride, N-acetylgalactosamine, mannosamine sulfate, mannosamine hydrochloride and N-acetylmannosamine and salts thereof.

6. The chemical complex according to claim 3, wherein said aminosugar derivative of a mono-saccharide is glucosamine sulfate or a salt thereof.

7. The chemical complex according to claim 1, wherein R′ and R″ are independently selected from optionally substituted C₁-C₁₀ alkyl, optionally substituted C₁-C₁₀ alkoxyl and from optionally substituted C₂-C₁₀alkenyl.

8. The chemical complex according to claim 1, wherein R′ and R″ are independently selected from optionally substituted C₁-C₆ alkyl, optionally substituted C₁-C₆ alkoxyl and from optionally substituted C₂-C₆ alkenyl.

9. The chemical complex according to claim 1, wherein R′ and R″ are independently selected from optionally substituted C₁-C₄ alkyl, optionally substituted C₁-C₄ alkoxyl and from optionally substituted C₂-C₄ alkenyl.

10. The chemical complex according to claim 1, wherein the one or more optionally substituted pyridine carboxy derivative is selected from the group constisting of niacinamide, thioniacinamide, 6-aminoniacinamide, N2-methyl-niacinamide, N2-ethylniacinamide, nicotinic acid, inosital hexaniacinate 6-methoxy-niacinamide and salts thereof.

11. The chemical complex according to claim 1, wherein the one or more optionally substituted pyridine carboxy derivative is selected from the group constisting of niacinamide, thioniacinamide, 6-aminoniacinamide, N2-methyl-niacinamide, N2-ethylniacinamide and salts thereof.

12. The chemical complex according to claim 11, wherein the one or more optionally substituted pyridine carboxy derivative is niacinamide or a salt thereof.

13. The chemical complex according to claim 1, wherein the one or more optionally substituted pyridine carboxy derivative(s) or salt(s) thereof and the one or more optionally substituted aminosugar(s) or salt(s) thereof are present in a molar ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

14. The chemical complex according to claim 1, wherein the one or more optionally substituted pyridine carboxy derivative(s) or salt(s) thereof and the one or more optionally substituted aminosugar(s) or salt(s) thereof are present in a mass ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

15. A composition comprising:
i) one or more optionally substituted pyridine carboxy derivative(s) or salt(s) thereof according to formula I; 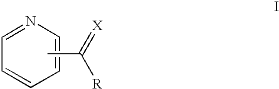
wherein X is selected from O and S; R is selected from OH; OR′; NH₂; NHR′; NR′R″, O^{-Y}⁺, and halogen, wherein R′ and R″ are independently selected from optionally substituted C₁-C₂₀ alkyl, optionally substituted C₁-C₂₀ alkoxyl and from optionally substituted C₂-C₂₀ alkenyl; and Y is a base addition salt of the free carboxylate; and
ii) one or more optionally substituted aminosugar(s) or salt(s) thereof; and
iii) one or more acceptable excipient(s) or carrier(s),
wherein the one or more optionally substituted aminosugar(s) is/are aminosugar derivative(s) of a mono-saccharide or an oligo-saccharide containing of at the most of six saccharide units.

16. The composition according to claim 15, wherein the one or more optionally substituted aminosugar(s) is/are aminosugar derivative(s) of a mono-saccharide or a di-saccharide.

17. The composition according to claim 15, wherein the one or more optionally substituted aminosugar(s) is/are aminosugar derivative(s) of a mono-saccharide.

18. The composition according to claim 15, with the proviso that said composition does not further comprise a source of phenyl alanine.

19. The composition according to claim 15, with the proviso that said composition does not further comprise vitamin C.

20. The composition according to claim 17, wherein said aminosugar derivative of a mono-saccharide is selected from the group consisting of glucosamine, galactosamine or mannosamine, their derivatives and salts thereof.

21. The composition according to claim 20, wherein said aminosugar derivative of a mono-saccharide is selected from the group consisting of glucosamine sulfate, glucosamine hydrochloride, N-acetylglucosamine, galactosamine sulfate, galactosamine hydrochloride, N-acetylgalactosamine, mannosamine sulfate, mannosamine hydrochloride and N-acetylmannosamine and salts thereof.

22. The composition according to claim 20, wherein said aminosugar derivative of a mono-saccharide is glucosamine sulfate or a salt thereof.

23. The composition according to claim 15, wherein R′ and R″ are independently selected from optionally substituted C₁-C₁₀ alkyl, optionally substituted C₁-C₁₀ alkoxyl and from optionally substituted C₂-C₁₀alkenyl.

24. The composition according to claim 15, wherein R′ and R″ are independently selected from optionally substituted C₁-C₆ alkyl, optionally substituted C₁-C₆ alkoxyl and from optionally substituted C₂-C₆ alkenyl.

25. The composition according to claim 15, wherein R′ and R″ are independently selected from optionally substituted C₁-C₄ alkyl, optionally substituted C₁-C₄ alkoxyl and from optionally substituted C₂-C₄ alkenyl.

26. The composition according to claim 15, wherein the one or more optionally substituted pyridine carboxy derivative is selected from the group constisting of niacinamide, thioniacinamide, 6-aminoniacinamide, N2-methyl-niacinamide, N2-ethyl-niacinamide, nicotinic acid, inosital hexaniacinate 6-methoxy-niacinamide and salts thereof.

27. The composition according to claim 15, wherein the one or more optionally substituted pyridine carboxy derivative is selected from the group constisting of niacinamide, thioniacinamide, 6-aminoniacinamide, N2-methyl-niacinamide, N2-ethyl-niacinamide and salts thereof.

28. The composition according to claim 15, wherein the one or more optionally substituted pyridine carboxy derivative is niacinamide or a salt thereof.

29. The composition according to 15, wherein the one or more optionally substituted pyridine carboxy derivative(s) or salt(s) thereof and the one or more optionally substituted aminosugar(s) or salt(s) thereof are present in a molar ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

30. The composition according to claim 15, wherein the one or more optionally substituted pyridine carboxy derivative(s) or salt(s) thereof and the one or more optionally substituted aminosugar(s) or salt(s) thereof are present in a mass ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

31. The composition according to claim 15 comprising:
i) a chemical complex as defined in any one of claims 1 to 14; and optionally
ii) one or more acceptable excipient(s) or carrier(s).

32. The composition according to claim 15 formulated as a pharmaceutical composition for oral, topical, transdermal, or parenteral administration.

33. The composition according to claim 32 formulated for oral or topical administration.

34. The composition according to claim 32 formulated for topical administration.

35. The composition according to claim 34 in solid or semi-solid form.

36. The composition according to claim 35, wherein the solid or semi-solid form is selected from the group consisting of pastes, ointments, hydrophilic ointments, creams, gels, hydrogels, lotions, and powders.

37. The composition according to claim 34 in liquid form.

38. The composition according to claim 37, wherein in the liquid form is selected from the group consisting of solutions, emulsions, suspensions, liniments and foams.

39. A method for suppression of hypersensitivity and suppression of inflammatory reactions in a mammal, comprising the administration to said mammal of an effective amount of a combination of one or more optionally substituted pyridine carboxy derivative(s) or salt(s) thereof and one or more optionally substituted aminosugar(s) or salt(s) thereof, or a chemical complex comprising said combination.

40. The method according to claim 39, wherein the suppression of hypersensitivity and/or suppression of inflammatory reactions is/are for the treatment of a dermatological disorder or disease.

41. The method according to claim 40, wherein the dermatological disorder or disease is selected from the group consisting of atopic dermatitis, contact dermatitis, seborrhoeic dermatitis, pruritus, nodular prurigo (prurigo nodularis hyde), senile prurigo, urticaria, acne, rosacea, alopecia, vitiligo and psoriasis.

42. The method according to claim 39, wherein the suppression of hypersensitivity and/or suppression of inflammatory reactions is/are for the treatment of a rheumatic disorder or disease.

43. The method according to claim 41, wherein the dermatological disorder or disease is selected from the group consisting of rheumatoid arthritis, osteoarthritis, ankylosing spondylitis, Reiter's syndrome, psoriastic arthritis, gout, juvenile chronic arthritis, enteropathic synovitis, infective arthritis, soft tissue rheumatism and fibromyalgia.

44. The method according to claim 39, wherein the suppression of hypersensitivity and/or suppression of inflammatory reactions is/are for chondroprotection or repair of articular cartilage.

45. The method according to claim 39, wherein the suppression of hypersensitivity and/or suppression of inflammatory reactions is/are for the treatment of IgE mediated allergic reactions

46. The method according to claim 39, wherein the suppression of hypersensitivity and/or suppression of inflammatory reactions is/are for the treatment of diseases and disorders selected from the group consisting of asthma, allergic rhinitis, allergic conjunctivitis and anaphylaxis.

47. The method according to claim 39, wherein the suppression of hypersensitivity and/or suppression of inflammatory reactions is/are for the treatment of an autoimmune disease and/or a chronic inflammatory disease.

48. The method according to claim 39, wherein the suppression of hypersensitivity and/or suppression of inflammatory reactions is/are for the treatment of diseases and disorders selected from the group consisting of diabetes, Crohn's disease, lupus erythematosus, Scieroderma, Sjögren's syndrome, Graves' disease, Pernicious anemia, autoimmune hepatitis, pemphigus vulgaris, pemphigus foliaceus, bullous pemphigoid, Myasthenia gravis and rheumatoid arthritis.

49. The method according to claim 39, wherein the one or more optionally substituted pyridine carboxy derivative(s) or salt(s) thereof and one or more optionally substituted aminosugar(s) or salt(s) thereof are together comprised in a single formulation or are each individually comprised in separate formulations.

50. The method according to claim 49, wherein the separate formulations are administered in a simultaneous or non-simultaneous manner.

51. The method according to claim 49, wherein the one or more optionally substituted pyridine carboxy derivative(s) or salt(s) thereof and one or more optionally substituted aminosugar(s) or salt(s) thereof are together comprised in a single formulation.

52. The method according to any of claim 51, wherein the single formulation or separate formulations are administered by means of oral, topical, transdermal, or parenteral administration, or combinations thereof.

53. The method according to claim 51, wherein the single formulation or separate formulations is administered by means of oral administration.

54. The use according to claim 51, wherein the single formulation or separate formulations is administered by means of topical administration.
